(19) United States
(12) Patent Application Publication
(10) Pub. No.: US 2021/0196251 A1
(43) Pub. Date: Jul. 1, 2021

(54) MEDICAL INSTRUMENT WITH SHAFT ACTUATING HANDLE CONFIGURED TO ACCEPT STYLET

(71) Applicant: Auris Health, Inc., Redwood City, CA (US)

(72) Inventors: Douglas Bruce Dull, San Jose, CA (US); Casey Teal Landey, San Francisco, CA (US); Ryan Jeffrey Connolly, San Carlos, CA (US); Marcus Andrew Foley, San Jose, CA (US)

(21) Appl. No.: 17/125,320

(22) Filed: Dec. 17, 2020

Related U.S. Application Data
(60) Provisional application No. 62/955,279, filed on Dec. 30, 2019.

(51) Int. Cl. A61B 10/02; A61M 25/01

(52) U.S. Cl. CPC A61B 10/0283; A61M 25/0102; A61B 1/2676; A61M 2205/0266; A61M 25/0136

(57) ABSTRACT

Medical instruments with shaft actuating handles configured to accept stylets are disclosed. In one aspect, a medical instrument includes an elongate channel having a distal end, a tool configured to be extended from and retracted into the distal end of the elongate channel, and a handle configured to drive movement of the tool between a retracted position and an extended position. The handle includes a casing, a handle member coupled to the tool and configured to move with respect to the casing as the tool is moved between the retracted position and the extended position, a fluid fitting coupled to the casing, and a flexible tube connecting the fluid fitting to the handle member and forming a portion of a lumen. The flexible tube is configured to allow a stylet to pass through the lumen when the tool is in the extended position.

### CROSS-REFERENCE TO RELATED APPLICATION(S)

This application claims the benefit of U.S. Provisional Application No. 62/955,279, filed Dec. 30, 2019, which is hereby incorporated by reference in its entirety.

### TECHNICAL FIELD

The present disclosure relates generally to surgical devices, and more particularly to a handle for actuating extension and retraction of a remotely-disposed tool via a shaft coupled between the handle and the tool.

### BACKGROUND

Endoscopy (e.g., bronchoscopy) may involve accessing and visualizing the inside of a patients airways for diagnostic and/or therapeutic purposes. During a bronchoscopy procedure a flexible tubular tool, known as a bronchoscope, may be inserted into the patient's nose or mouth and passed down the patient's throat into the lung airways towards a tissue site identified for subsequent diagnosis and/or treatment. The bronchoscope can have an interior lumen (a “working channel”) providing a pathway to the tissue site, and catheters and various medical tools can be inserted through the working channel to the tissue site.

### BRIEF DESCRIPTION OF THE DRAWINGS

The disclosed aspects will hereinafter be described in conjunction with the appended drawings and appendices, provided to illustrate and not to limit the disclosed aspects, wherein like designations denote like elements.

FIGS. 1A-1F illustrate an embodiment of a medical instrument including a shaft manipulating handle as described herein.

FIGS. 2A-2D illustrate another embodiment of a shaft manipulating handle as described herein.

FIGS. 3A and 3B illustrate photos of an example shaft manipulating handle as described herein.

FIGS. 4A-4H illustrate another embodiment of a shaft manipulating handle as described herein.

FIGS. 5A-5E illustrate various alternate handle embodiments

FIG. 6 depicts a schematic diagram of a robotic surgical system for actuating a handle as described herein.

FIG. 7 depicts a flowchart of an embodiment of a process for driving movement of a medical instrument using a handle as described herein.

FIGS. 8A and 8B illustrate cutaway views of a proximal end of a handle, such as the handle of the instrument illustrated in FIGS. 1A-1F.

FIGS. 9A-9C illustrate cutaway views of another embodiment of a proximal end of a handle, such as a modified embodiment of the handle of the instrument illustrated in FIGS. 1A-1F in accordance with aspects of this disclosure.

FIGS. 10A and 10B illustrate cutaway views of an embodiment of a fluid fitting, such as the fluid fitting of FIGS. 8A and 8B.

FIGS. 11A and 11B illustrate cutaway views of another embodiment of a fluid fitting, such as the fluid fitting of FIGS. 9A-9C.

### DETAILED DESCRIPTION

### Introduction

Medical procedures may involve manipulation of a tool positioned remotely from the operator, for example positioned through a channel inserted into the body of a patient. Such channels include trocars, catheters, and endoscopes including bronchoscopes. As one example of such a medical procedure, transbronchial needle aspiration (TBNA) can be used as a minimally invasive bronchoscopic technique for diagnosis and staging of bronchial diseases, including lung cancer. A TBNA technique can involve manipulating a biopsy needle through a flexible bronchoscope. For example, a physician can use chest scans to identify the location of a mass to be biopsied and to guide positioning of the bronchoscope within the patient's airways towards that mass. After the distal end of the bronchoscope working channel is positioned within the airways near the identified mass, an elongate, tubular jacket containing the biopsy needle can be advanced through the working channel to the sampling area. The target tissue can then be pierced by extending the needle out of the jacket, and aspiration can be applied to aid sample acquisition. Typically, sample acquisition involves holding the proximal end of a tube attached to the needle by hand and manually moving the tube backward and forward relative to the bronchoscope to repeatedly puncture the tissue site with the needle. A vacuum (e.g., provided by a syringe or other aspiration or respiration device) can be used to provide aspiration in order to aid in drawing target tissue into the distal end of the needle. After sample acquisition, the needle can be retracted back into the sheath and withdrawn through the working channel.

In some procedures, sample analysis can be performed in the same room as the TBNA procedure, and depending upon results of the analysis further TBNA sample acquisition(s) and/or other tissue sampling or treatment can be performed. In order to perform analysis on the extracted samples, the samples can be expelled from the distal end of the needle by applying pressure to the samples through the needle, for example, by using the syringe or other pressure source. However, the samples acquired during a tissue sampling procedure can occasionally become lodged or otherwise stuck in the needle (e.g., due to the tissue samples being tightly packed in the needle) such that the pressure provided by the syringe is insufficient to expel the samples from the distal end of the needle. Aspects of this disclosure relate to a medical instrument which can expel acquired tissue samples using an alternate or auxiliary technique.

Bronchoscopy techniques including TBNA can have difficulty accessing masses at the periphery of the lungs, particularly if such masses are still relatively small, for example around 8 mm or smaller. This limitation can, in some instances, prevent successful use of bronchoscopy in diagnosing and staging cancerous masses in early stages, a timeframe during which such masses may be more easily treatable and may not have spread to other places in the patient's body. Another consideration with bronchoscopy at the lung periphery relates to the risk of pneumothorax if the needle (or other tool) is not carefully controlled and thus pierces the lung. Further, existing bronchoscopy systems usable for TBNA and other airway sampling and treatment techniques require multi-handed operation, often involving multiple people to position and maintain the bronchoscope and then to actuate movement of instruments through the bronchoscope working channel.

The aforementioned considerations, among others, are addressed in some embodiments by the actuating handles described herein. Embodiments of the disclosure relate to actuating handles, specifically handles for actuating extension and retraction of a medical tool disposed remotely from the handle via a shaft coupled between the handle and the tool. Further, the described handles provide multiple modalities for moving such tools. The handle can include mechanisms that permit control of linear motion of the shaft secured within the handle, for example including one or both of a rotational interface and a plunging interface. The rotational interface can allow for fine-control positioning of the shaft, for example by allowing a user to rotate the rotational interface to extend or retract the shaft. Some implementations of the rotational interface can include detents to provide physical feedback (e.g., haptic feedback) to the user regarding when the rotation has caused a certain interval of the extension or retraction. The plunging interface can enable a faster linear motion, for example by implementing a biasing mechanism such as a spring that compresses during retraction of the shaft and then releases to drive rapid linear motion in the extension direction.

Thus, the disclosed handle can provide enhanced control of the medical tool through the multiple motion-transmitting interfaces, and can be sized such that both interfaces are capable of use by a single hand. Beneficially, this can allow a physician to actuate tool motion themselves during an endoscopic procedure without requiring another person to assist.

In the context of use with a bronchial tool, the rotational interface can be structured to provide sufficient extension of the tool to reach the lung periphery from the distal end of the working channel, thus improving access to previously inaccessible masses peripheral lung regions. Further in the context of use with a bronchial tool, the rotational interface can be structured to only allow extension of the tool tip by a specified amount that is equal to or slightly smaller than (e.g., several millimeters) the expected distance between the working channel distal end and the outer edge of the lung. For example, some bronchoscope systems can position the working channel distal end approximately 2.5 cm to 3 cm from the outer edge of the lung. Thus, a handle designed for use with such a system can limit needle extension to 2.5 cm or 3 cm in order to enable biopsy of a mass at the lung periphery while reducing the risk of pneumothorax. It will be appreciated that this specific distance is provided for example only, and that different handles according to the present disclosure can be made to provide specific extension distances corresponding to specific bronchoscopes. Further, the plunging interface can beneficially be designed in such embodiments to withdraw the needle or tool from the tissue site so that the maximum extension distance of the rotational interface is not exceeded. Releasing the tool back into the tissue site with force via the biasing element of the plunging interface can beneficially aid in collection of tissue samples in some embodiments.

A handle, according to the present disclosure, can be provided for an elongate medical instrument designed to be operated through a working channel positioned in a body cavity of a patient. The medical instrument can have an elongate shaft, tube, or wire coupled to a tool. For example, the instrument can be a flexible sheath containing biopsy needle coupled to a distal end of a tube, with the needle positioned at a distal end of the flexible sheath. Additional examples of tools that can be used with the disclosed handles include brushes (e.g., a cytology brushes), needle-tipped cytology brushes, forceps (e.g. biopsy forceps), baskets, bone biopsy needles, fiducial markers and their delivery mechanisms, diathermy snares, laproscopic tools, angioplasty balloons, stents, or other endoscopic or catheter-delivered or catheter-based medical instruments or tools.

As used herein, “distal” refers to the end of the scope or tool positioned closest to the patient tissue site during use, and “proximal” refers to the end of the sheath or tool positioned closest to the operator (e.g., a physician or a robotic control system). Stated differently, the relative positions of components of the sheath, tool, and/or the robotic system are described herein from the vantage point of the operator.

Thus, as used herein, a “remotely-disposed” tool refers to the tool being located at or beyond the distal end of a working channel with the handle being located at or beyond the proximal end of the working channel. The term remotely-disposed can also refer to tools that are not inserted through any working channel but are separated from the handle by a distance spanning an elongate jacket containing the tool, for example a catheter positioned through a blood vessel or other luminous passage of a patient.

As used herein, a biasing element can be one or more of a spring, opposing magnets, hydraulic systems, compressible shape memory alloys, and other mechanisms that can store potential energy in compression or extension and then effect movement due to release of the potential energy during the other of extension or compression.

As used herein, the term “dithering” refers to a back and forth motion of a medical instrument such as a biopsy needle, for example by extension and retraction of the instrument using the plunging interface of the handle described herein. In some cases, the back and forth motion of the medical instrument occurs independent of the movement of the instrument's jacket such that the jacket of the medical instrument remains relatively stationary during the dithering.

The disclosed systems and techniques can provide advantages for bronchoscopic needle biopsies and other applications, including manipulation of other endoscopic, laparoscopic, and/or catheter-delivered tools. Thus, though the disclosed handles are described in many portions of the present disclosure within the context of bronchoscopy biopsy needles, it should be understood that such handles can also be used with other remotely-disposed tools in order to provide the disclosed benefits. Further, though the disclosed handle is illustrated and described with both the plunging and rotational interfaces, it will be appreciated that alternatives can include one of these interfaces without the other, and that the plunging features from the various embodiments described herein can be combined with the rotational features from other embodiments described herein.

Robotic surgical systems can utilize endoscopic instruments to perform minimally invasive endoscopic procedures robotically. Thus, some implementations of the disclosure relate to surgical instruments and systems that include shaft actuation handles that can advantageously be used in robotically-guided (whether fully automated robotic systems or robotic systems that provide some level of assistance) medical procedures, and methods of performing a medical procedure under guidance of a robotic surgical system. In such systems, a robotic arm can be configured to control the rotation and plunging motions described herein. Drive signals for such actuation can be supplied by the robotic surgical system, for example in response to user input via an input device and/or computer-controlled surgical processes.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### Overview of Example Handles

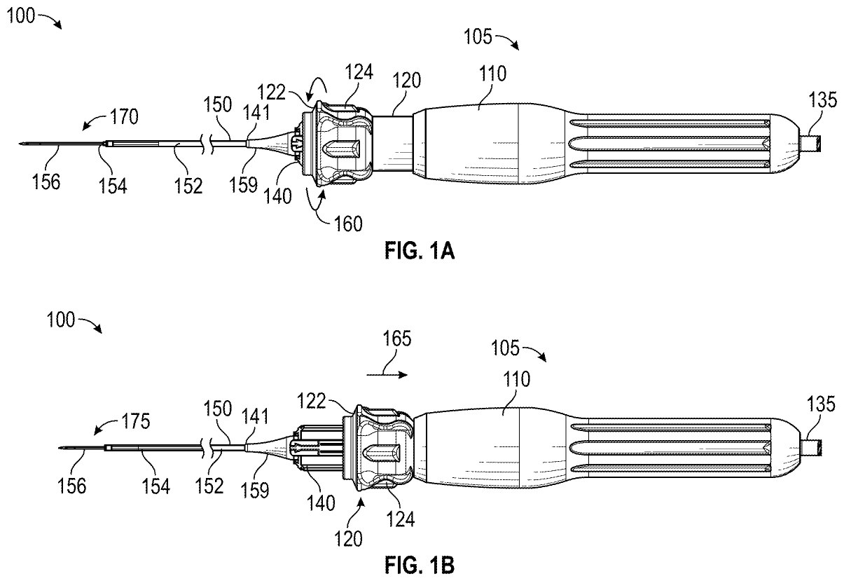

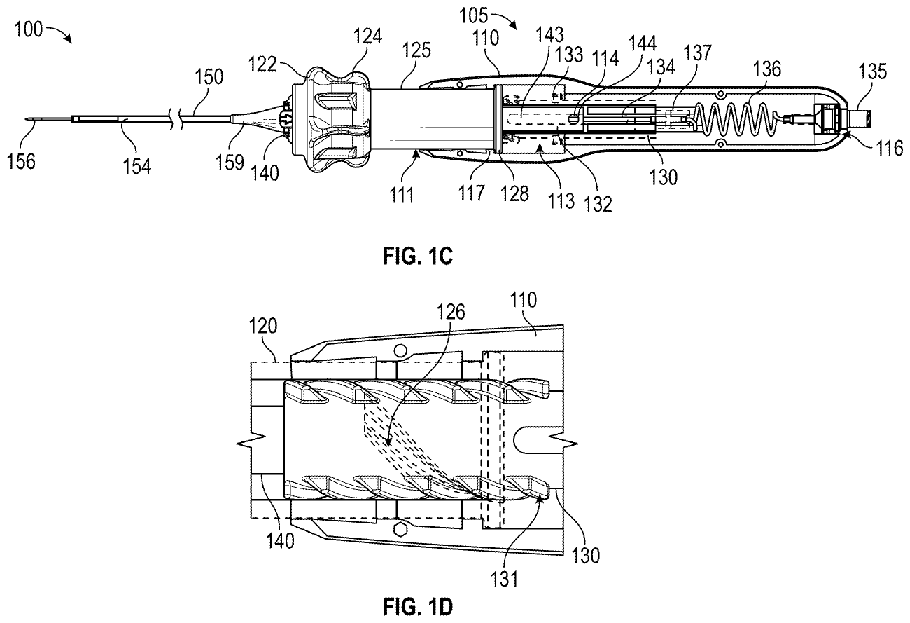

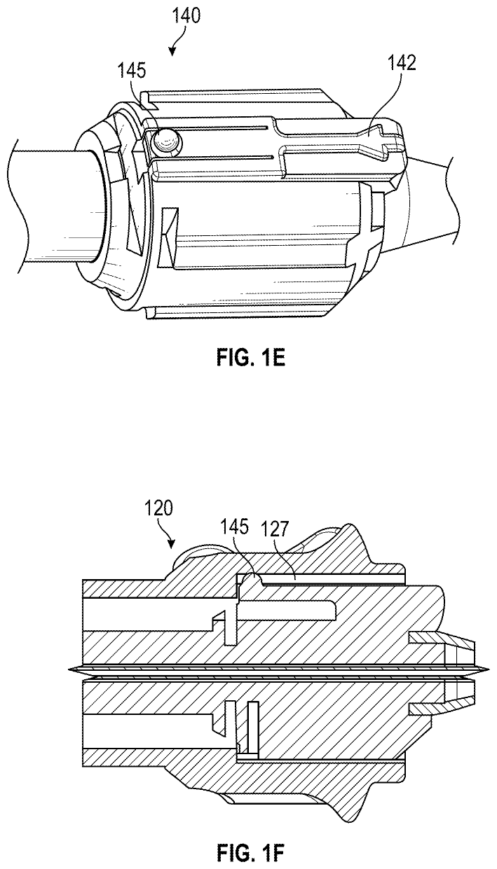

FIGS. 1A-1F illustrate an embodiment of a medical instrument 100 including a shaft manipulating handle 105, jacket 150, conduit 154, and tool 156. FIG. 1A illustrates an outer view of the instrument 100 and the rotational 160 modality. FIG. 1B illustrates an outer view of the instrument 100 and the plunging 165 modality. FIG. 1C illustrates a cutaway view of the handle 105 of the instrument 100. FIG. 1D illustrates a cutaway view of a portion of the handle 105 with the actuation sleeve 120 shown with reduced opacity to reveal interior structures. FIG. 1E illustrates an outer view of the distal handle member 140, and FIG. 1F illustrates a cross-sectional view of the distal handle member 140 within the actuation sleeve 120. FIGS. 1A-1F are discussed together in portions of the description below due to the overlap of depicted features.

With reference to FIG. 1A, the handle 105 includes an actuation sleeve 120, distal handle member 140, casing 110, and fluid fitting 135. The actuation sleeve 120 includes a rotational wheel grip 124 and a plunger grip 122. As shown in FIG. 1A, an operator can drive motion of the tool 156 relative to the jacket 150 by rotating 160 the rotational wheel grip 124, which causes rotation of the actuation sleeve 120 around the longitudinal axis of the handle 105. Rotation 160 in one direction can cause extension of the tool 156 from the jacket 150. Rotation in the other direction can retract the tool 156 back into the jacket 150. The internal components of the handle 105 that transfer this motion to the tool 156 are described in more detail below. As described in more detail below, the components of the handle 105 can be structured to provide physical feedback to the user at predetermined intervals to assist in fine control of the tool extension.

The plunging modality, as illustrated in FIG. 1B, can be driven in one direction by an application of force by the operator to plunger grip 122 and driven in the opposite direction by a biasing element upon release of the force. The biasing element and internal components of the handle the handle 105 that transfer this motion to the tool 156 are discussed in more detail below. When the actuation sleeve 120 is drawn proximally as shown by arrow 165 in FIG. 1B, for example by application of pressure to the plunging surface 122, the biasing element can be compressed. Upon release of at least some of the pressure from the plunging grip 122 the biasing element can expand, thereby driving distal motion of the actuation sleeve 120. In the embodiment of FIGS. 1A and 1B, during rotation 160 and plunging 165 the fluid fitting 135 may remain stationary with respect to the casing 110 of the handle.

Due to the biasing element, the plunging modality can be useful for effecting a dithering motion of the tool. For example, the tool can be extended to a desired maximum distance using the rotational modality, such as a desired distance into patient tissue. By applying pressure to the plunging grip 122, an operator can retract the actuation sleeve, thereby driving retraction of the tool proximally towards the operator and away from the patient tissue. In some embodiments, complete retraction of the actuation sleeve 120 can retract the tool by approximately 1.5 cm, approximately 2 cm, or another desired distance. Thus, the plunging motion may not cause further extension of the tool. This can be beneficial during use in pulmonary procedures near the lung periphery to mitigate the risk of pneumothorax. Dithering of a tool such as a biopsy needle, which refers to the repeated retraction and extension as can be caused by multiple uses of the plunging modality, can assist in acquisition of a suitable tissue sample.

The jacket 150, conduit 154, and tool 156 of the medical instrument are illustrated together with the handle 105 in FIGS. 1A-1C. As illustrated, the jacket 150 extends from a distal aperture 141 of the handle 105 through strain relief 159, and can contain some or all of the conduit 154 and tool 156 in various configurations. The tool 156 is depicted as a needle and the conduit 154 has an interior lumen 152 that provides at least a portion of a fluid pathway between a proximal aperture of the fluid coupling 135 of the handle 105 and the distal end of the tool 156. The tool 156 can be a biopsy needle such as an aspiration needle configured for acquisition of tissue samples or can be configured for delivery of therapeutic agents to a tissue site. In other examples, the tool 156 can be a brush (e.g., a cytology brush), a needle-tipped cytology brush, forceps (e.g. biopsy forceps), or other tools as described above. Where aspiration is not required with a particular tool, the conduit may have no interior lumen and instead may have a solid cross-section or be composed of braided strands.

The views of FIGS. 1C and 1D show internal components of the handle 105. As shown in FIG. 1C, the handle 105 includes a casing 110, actuation sleeve 120, proximal handle member 130, and distal handle member 140. These components can be printed, molded, or machined from suitable materials including plastics, metals, and composites. Extending from a distal aperture 141 of the handle 105 is a flexible, tubular jacket 150. A strain relief 159 may be fitted with the distal end of the handle 105 around the jacket 150. A conduit 154 is positioned within the interior lumen of the jacket 150 and coupled to a medical tool 156 at its distal end and to the proximal handle member 130 at its proximal end. As such, linear motion of the proximal handle member 130 along a longitudinal axis of the handle 105 via one or both of the rotational and plunging modalities described herein can drive corresponding motion of the conduit 154, thus driving extension and retraction of the tool relative to the distal end of the jacket 150.

The casing 110 can provide an internal volume to enclose at least a portion of the moving parts of the handle 105, and can provide an external surface sized and shaped to provide a comfortable surface for grasping with a single hand in some implementations. For example, a user can hold a portion of the casing 110 in the palm and heel of the hand while manipulating the actuation sleeve 120 with the fingers, thereby allowing the user to control extension and retraction of the tool 156 with a single hand. The casing 110 can include a distal aperture 111 at its distal end and a proximal aperture 116 at its proximal end.

A portion of the internal volume of the casing can provide a housing 113 for constraining the range of motion of the actuation sleeve 120. This housing 113 can have a larger diameter than a distal portion of the internal volume in some embodiments in order to provide an annular surface for engaging the flange 128 of the actuation sleeve at a fully retracted position of the actuation sleeve 120. The flange 128 of the actuation sleeve can abut a flange 117 of the casing 110 at a fully extended position. The flange 117 can serve to limit the extension of the actuation sleeve 120 relative to the casing 110. Thus, in some embodiments the length of the housing 113 can be selected to correspond to the desired range of motion of the actuation sleeve 120. In some embodiments the positioning of flange 117 along the longitudinal axis of the handle 105 may not be fixed, and an operator can adjust its positioning to provide control over the desired plunging distance.

The housing 113 can additionally contain a spring or other biasing element, for example a pair of opposing magnets, a chamber of compressible hydraulic fluid, or a shape memory alloy. The annular proximal surface of the housing 113 can engage a proximal portion of the biasing element, and a distal portion of the biasing element can engage the flange 128 of the actuation sleeve 120. In some embodiments, when the actuation sleeve 120 is drawn proximally as shown by arrow 165 in FIG. 1B, the biasing element can be compressed by proximal movement of the flange 128 of the actuation sleeve 120. Upon release of the pressure on the plunging grip the biasing element will expand and drive the actuation sleeve 120 distally until flange 128 abuts flange 117 of casing 110, thereby driving the tool back to its extended position.

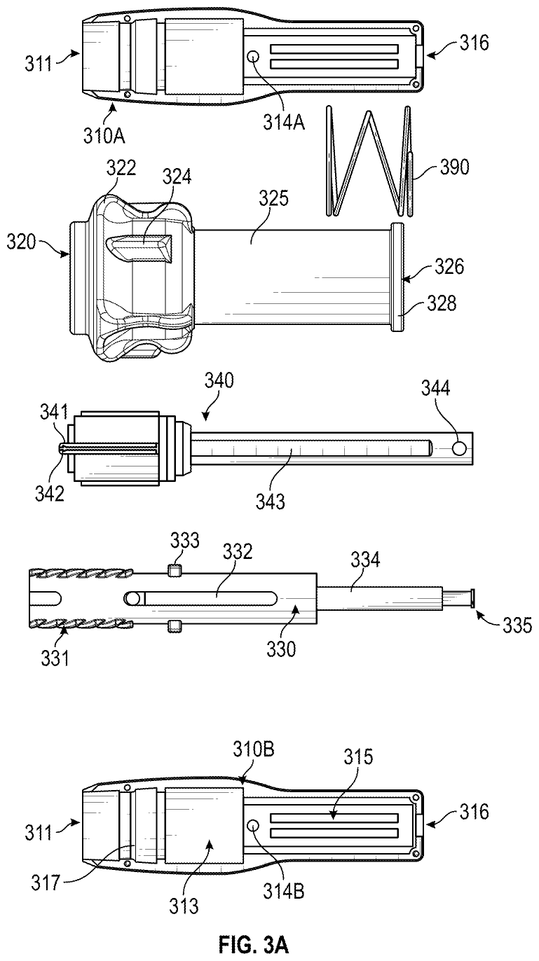

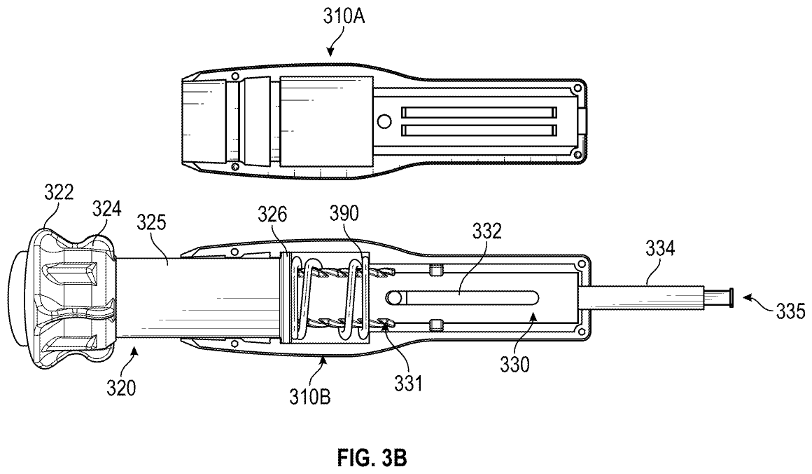

The casing 110 can also include at least one prong 114 positioned to extend into the aperture 144 of the distal handle member 140 to secure the distal handle member 140 relative to the casing 110. The prong extending of the portion of casing 110 shown in FIG. 1C is positioned behind the conduit 154, and for reference a similar prong 314A, 314B are shown in FIG. 3A. The prong 114 can have a length sufficient to be secured within the aperture 144 of the distal handle member 140 without occluding the lumen through which conduit 154 passes. As such, the prong 114 can have a length of less than half the diameter of the internal volume of the casing 110.

The actuation sleeve 120 can have a proximal flange 128, a cylindrical body 125 extending from the flange 128 to a rotational wheel grip 124, plunger grip 122, and an internal cam interface (shown and described in actuation sleeve of FIG. 1D). Rotational wheel grip 124 can be used to facilitate the rotational modality described and the plunger grip 122 can be used to facilitate the plunging modality as described above. For example, rotational wheel grip 124 can provide a number of grip surfaces extending radially from the distal end of the actuation sleeve. Plunger grip 122 can provide a surface that allows a user to exert force on a portion of the actuation sleeve 120 to draw actuation sleeve 120 proximally, and can be formed for example by a distal surface of the rotational wheel. The cylindrical body 125 can be sized to slide through the distal aperture 111 of the casing 110 during manipulation of the actuation sleeve 120 within the casing 110, and the flange 128 can abut the flange 117 of the casing 110 to provide a mechanical stop for forward extension of the actuation sleeve 120.

The proximal handle member 130 can include, from its proximal end towards its distal end: fluid fitting 135, proximal portion 136, recess 137, elongate slot 132, support annulus 133, and external cam interface 131. A portion of the external cam interface 131 is visible in FIG. 1C, and its interaction with an internal cam interface. An internal cam interface and the interaction between the external cam interface 131 and the internal cam interface is discussed in more detail below with respect to FIG. 1D.

The conduit 154 attached to tool 156 can be secured within recess 137, for example by bonding via an adhesive. Thus, linear motion of the proximal handle member 130 can transfer to the tool 156 via the conduit 154, allowing manipulation of the handle 105 to drive extension and retraction of tool 156 from jacket 150. In some embodiments the recess 137 can be structured to mechanically mate with a corresponding feature on the conduit 154 to facilitate use of the handle 105 with a number of different conduits and tools. Thus, in some embodiments the handle 105 may be sterilizable and reusable while the conduit, tool, and jacket may be disposable. In various other embodiments the entire instrument 100 may be entirely sterilizable and reusable or designed as a disposable single unit.

Fluid fitting 135 can be a threaded connector for securing to a corresponding threaded connector of an aspiration device, a respiration device, or device containing therapeutic agents. In one example, the fluid fitting 135 can be a Luer lock. The fluid fitting 135 can be secured within the proximal aperture 116 of the casing 110 in some embodiments. Securing the fluid fitting 135 to the casing 110 can provide benefits in terms of stability of the aspiration device when secured to the fluid fitting 135.

As shown, the proximal portion 136 of proximal handle member 130 can comprise a length of coiled tubing in some implementations having the fluid fitting 135 fixed to the casing 110. This can provide a flexible fluid path that accommodates linear motion of the proximal handle member 130. For example, the proximal portion 136 can be coiled HDPE tubing, and, in some embodiments, this can be a portion of the conduit 154 positioned proximally from the bonding recess 137. A sleeve of polyolefin heat shrink can be used to secure the coiled tubing to the fluid fitting in some implementations.

Elongate slot 132 can have a length and width sufficient to allow the prong of the casing 110 that secures the distal handle member 140 to slide through the slot 132 as the proximal handle member 130 moves linearly within the casing 110. Support annulus 133 can have an outer diameter that substantially matches the inner diameter of a proximal portion of the interior volume of the casing 110 to slidably engage the inner wall of the casing and provide stability to the proximal handle member 130 during linear movement.

The distal handle member 140 can have a proximal shaft 143 positioned partially within the actuation sleeve 120 and partially within the proximal handle member 130. The proximal shaft 143 can have a recess or aperture 144 sized to accept the prong 114 of the casing 110, thereby fixing the position of the distal handle member relative to the casing 110.

Turning specifically to FIG. 1D, an internal cam interface 126 of the actuation sleeve 120 can be formed as grooves extending in a helical or spiral structure around the inner surface of the actuation sleeve 120, and external cam interface 131 can be formed as ridges extending in a helical or spiral structure around the exterior surface of the proximal handle member 130. In other embodiments the internal cam interface 126 can comprise ridges and the external cam interface 131 can comprise grooves. The external cam interface 131 can be positioned at least partly within the actuation sleeve 120. The external cam interface 131 can engage the internal cam interface 126 of the actuation sleeve 120 to form a motion transmitting interface for transmitting rotational or linear motion of the actuation sleeve 120 to the proximal handle member 130. Thus, in embodiments where the internal cam interface 126 comprises grooves, the external cam interface 131 can comprise ridges angled to engage the grooves. Similarly, in embodiments where the internal cam interface 126 comprises ridges, the external cam interface 131 can comprise grooves angled to be engaged by the ridges.

The engaged external and internal cam interfaces 131, 126 shown in FIG. 1D can transfer the rotational motion of the actuation sleeve 120 to linear motion of the proximal handle member 130. The engaged external and internal cam interfaces 131, 126 can transfer the linear plunging motion of the actuation sleeve 120 to the proximal handle member 130 and thus to the conduit 154 and tool 156 shown in FIGS. 1A-1C. Although not illustrated, some embodiments may provide a lock for the actuation sleeve 120 to prevent further rotation during the plunging modality. The bond between the conduit 154 and the proximal handle member 130 can, in turn, transfer this motion to the tool to drive the extension and/or retraction.

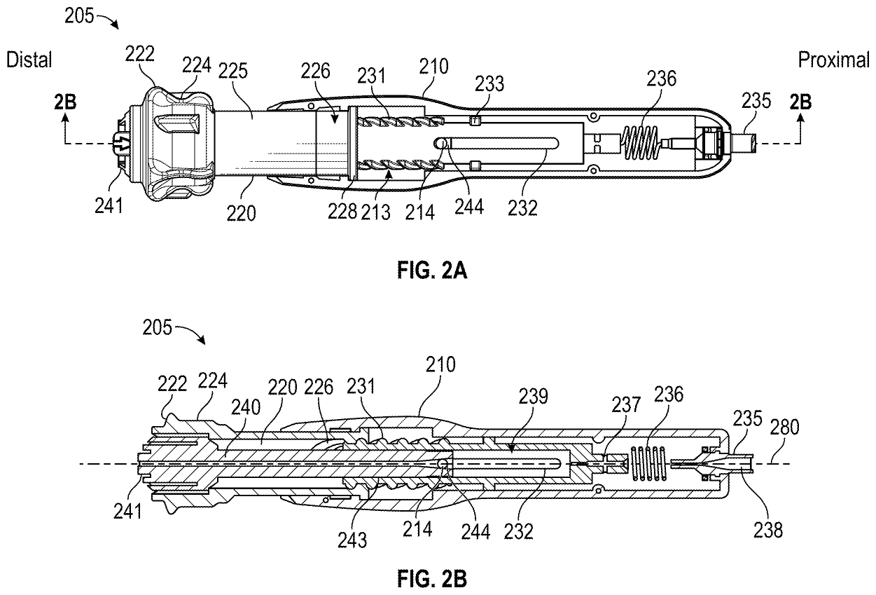

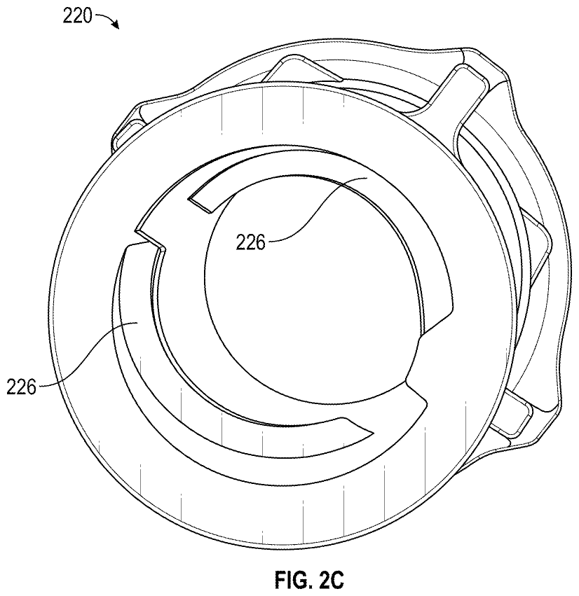

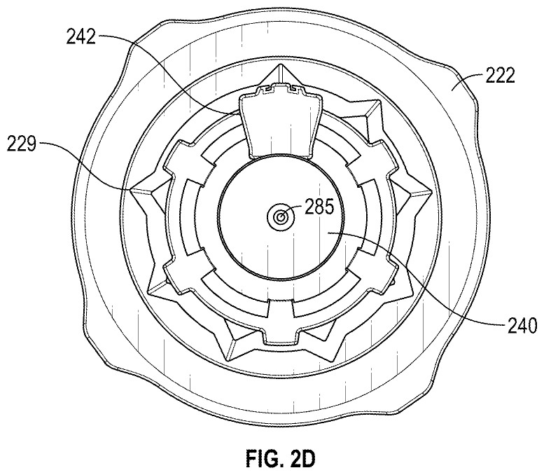

As shown in FIGS. 1E and 1F, the distal end of the distal handle member 140 can include a position indicator 142 and a rounded protrusion 145. The position indicator 142 can line up with extension distance markings on a distal surface of the actuation sleeve 120 (see FIG. 2C and associated description for an example) in order to provide an operator with a visual indication of how far the tool is extending from the jacket 150 based on the rotation of the actuation sleeve 120.

The rounded protrusion 145 can be sized to allow rotation of the actuation sleeve 120 around the rounded protrusion 145 while slightly compressing the rounded protrusion 145 inwardly into the distal handle member 140. The actuation sleeve 120 can have one or more detents 127 sized to receive the uncompressed rounded protrusion 145 to provide physical feedback to an operator when the actuation sleeve 120 has been rotated a specified amount and to gently lock the position of the actuation sleeve 120 relative to the distal handle member 140. The actuation sleeve 120 can include a number of such detents aligned with distance markings visible to the user. For example, the actuation sleeve 120 can provide a detent and marking at intervals corresponding to every 1, 2, 5, or 10 mm of extension or retraction of the tool. One example can be configured to provide up to 30 mm of extension and can have a detent corresponding to every 5 mm of extension.

FIGS. 2A-2D illustrate another embodiment of a shaft manipulating handle 205 as described herein. FIG. 2A illustrates a cutaway view of the casing 210 of the handle 205 to reveal the actuation sleeve 120 and proximal handle member 230 within, with the actuation sleeve 120 shown with a slight opacity reduction to reveal the internal cam interface 228. FIG. 2B illustrates a cross-sectional view of the handle 205. FIG. 2C illustrates a perspective view of the actuation sleeve 220 of the handle 205. FIGS. 2A-2D will be discussed together below. FIG. 2D illustrates a front view of the handle.

Similar to the handle 105 of FIGS. 1A-1F, the handle 205 can be operated in both rotational and plunging modalities for fine tool driving and dithering. As shown in FIGS. 2A-2C, the handle 205 includes a casing 210, actuation sleeve 220, proximal handle member 230, and distal handle member 240 positioned along a longitudinal axis 280. A lumen 285 (shown in FIG. 2D) can form a fluid pathway from the proximal end of the handle 205 to the distal end of the handle 205.

The casing 210 can provide an internal volume to enclose at least a portion of the moving parts of the handle 205, and can provide an external surface sized and shaped to provide a comfortable surface for grasping with a single hand in some implementations. The casing 210 can include a distal aperture at its distal end and a proximal aperture at its proximal end. The casing 210 can also include at least one prong 214 positioned to extend into the aperture 244 of the distal handle member 240 without occluding the handle lumen 285 in order to secure the distal handle member 240 relative to the casing 210. A portion of the internal volume of the casing can provide a housing 213 for constraining the range of motion of the actuation sleeve 220 and housing a biasing element.

Similar to actuation sleeve 120, actuation sleeve 220 can have a proximal flange 228, a cylindrical body 225 extending from the flange 228 to a rotational wheel grip 224, plunger grip 222, and an internal cam interface 226. Rotational wheel grip 224 can be used to facilitate the rotational modality described herein, and the plunger grip 222 can be used to facilitate the plunging modality described herein. FIG. 2C illustrates a perspective view showing example ridges forming the internal cam interface 226.

Similar to proximal handle member 130, the proximal handle member 230 can include, from its proximal end towards its distal end: fluid fitting 235, proximal portion 236, recess 237 for coupling with a tool conduit, elongate slot 232, support annulus 233, and external cam interface 231. Proximal portion 236 can comprise a flexible length of tubing as discussed above or can comprise a rigid shaft. If proximal portion 236 comprises a rigid shaft, the fluid fitting 235 can be at a proximal end of the rigid shaft and can move relative to the casing 210 during motion of the proximal handle member 230. As described above, the external cam interface 231 can be positioned at least partly within the actuation sleeve 220. The external cam interface 231 can engage the internal cam interface 226 of the actuation sleeve 220 to form a motion transmitting interface for transmitting rotational or linear motion of the actuation sleeve 220 to the proximal handle member 230.

Similar to the distal handle member 140, the distal handle member 240 can include a proximal shaft 243 positioned partially within the actuation sleeve 220 and partially within an internal receiving volume 239 of the proximal handle member 230. The proximal shaft 243 can have a recess or aperture 244 sized to accept the prong(s) 214 of the casing 210, thereby fixing the position of the distal handle member 240 relative to the casing 210. The distal handle member 240 can include distal aperture 241 through which the conduit secured to the proximal handle member 230 may extend.

FIG. 2D illustrates an example design for the distal end of the handle relating to markings for providing visual extension distance indicators. Position indicator 242 can line up with (or be positioned between) radially-spaced distance indicators 229 on the actuation sleeve 220. Initially, the tool coupled to handle 205 may be positioned with its distal tip at or near the distal end of a jacket. Rotation of the actuation handle 240 can cause controlled extension of the tool from the jacket, and the radially-spaced distance indicators 229 can provide visual indications of how far the distal tip of the tool is extended beyond the distal end of the jacket. Though illustrated as triangular configurations, other designs may use dots, lines, numerical markings, or a combination of these.

FIGS. 3A and 3B illustrate photos of an example shaft manipulating handle 305 as described herein. FIG. 3A illustrates a disassembled view of the handle 305. FIG. 3B illustrates an assembled view of the handle 305 with the first casing portion 310A removed to show the arrangement of the proximal handle member 330, spring 390, actuation sleeve 320, and distal handle member 340.

As shown in FIG. 3A, similar to handles 105 and 205, the handle 305 can include a casing, actuation sleeve 320, proximal handle member 330, distal handle member 340, and can also include the spring 390. The casing can be formed in first and second portions 310A, 310B that can secure together around the cylindrical body 325 of the actuation sleeve 320 and a distal portion of the proximal handle member 330.

The casing 310A, 310B can provide an internal volume to enclose at least a portion of the moving parts of the handle 305, and can provide an external surface sized and shaped to provide a comfortable surface for grasping with a single hand in some implementations. The casing 310A, 310B can include a distal aperture 311 at its distal end and a proximal aperture 316 at its proximal end. Each half of casing 310A, 310B can include a prong 314A, 314B positioned to extend into the aperture 344 of the distal handle member 340 without occluding a lumen extending through the handle 305. As described above, this can secure the positioning of the distal handle member 340 relative to the casing 410. A portion of the internal volume of the casing can provide a housing 313 for constraining the range of motion of the actuation sleeve 320 and for housing spring 390.

Similar to actuation sleeves 120, 220, actuation sleeve 320 can have a proximal flange 328, a cylindrical body 325 extending from the flange 328 to a rotational wheel grip 324, plunger grip 322, and an internal cam interface 326 (within the actuation sleeve 320 but not visible in FIG. 3A). Rotational wheel grip 324 can be used to facilitate the rotational modality described herein, and the plunger grip 322 can be used to facilitate the plunging modality described herein.

Similar to proximal handle members 130 and 230, the proximal handle member 330 can include, from its proximal end towards its distal end: fluid fitting 335, proximal portion 334, a fastener or fastening mechanism (not shown) for coupling with a tool conduit, a pair of elongate slots 332, support annulus 333, and external cam interface 331. Proximal portion 334 can comprise a rigid shaft having fluid fitting 335 at a proximal end of the rigid shaft. Thus, fluid fitting 335 can move relative to the casing 410 during motion of the proximal handle member 330 and the proximal portion 334 can be sized to pass through the proximal aperture 316 of the casing 310A, 310B. As described above, the external cam interface 331 can be positioned at least partly within the actuation sleeve 320. The external cam interface 331 can engage the internal cam interface 326 of the actuation sleeve 320 to form a motion transmitting interface for transmitting rotational or linear motion of the actuation sleeve 320 to the proximal handle member 330.

Similar to the distal handle members 140 and 240, the distal handle member 340 can include a proximal shaft 343 positioned partially within the actuation sleeve 320 and partially within the proximal handle member 330. The proximal shaft 343 can have an aperture 344 sized to accept the prongs 314A, 314B of the casing 310A, 310B, thereby fixing the position of the distal handle member 340 relative to the casing 310A, 310B as shown in FIG. 3B. The distal handle member 340 can include distal aperture 341 through which the conduit secured to the proximal handle member 330 may extend and can include rotation indicator 342.

Turning to FIG. 3B, the actuation sleeve 320, proximal handle member 330, distal handle member 340, and spring 390 are assembled with portion 310B of the casing in place and portion 310A open to show the interior configuration. FIG. 3B illustrates how the spring 390 can be secured within the housing 313 to bias the actuation handle 320 distally with flange 328 pressed against flange 317 of the casing.

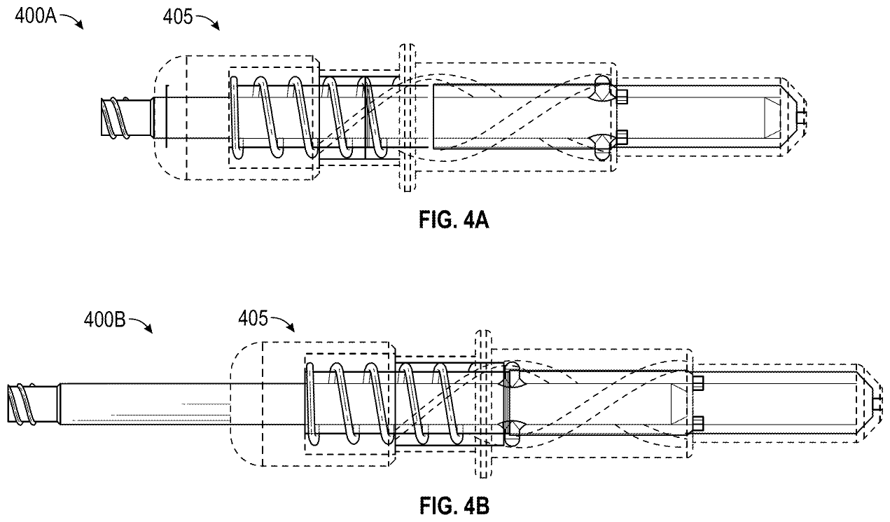

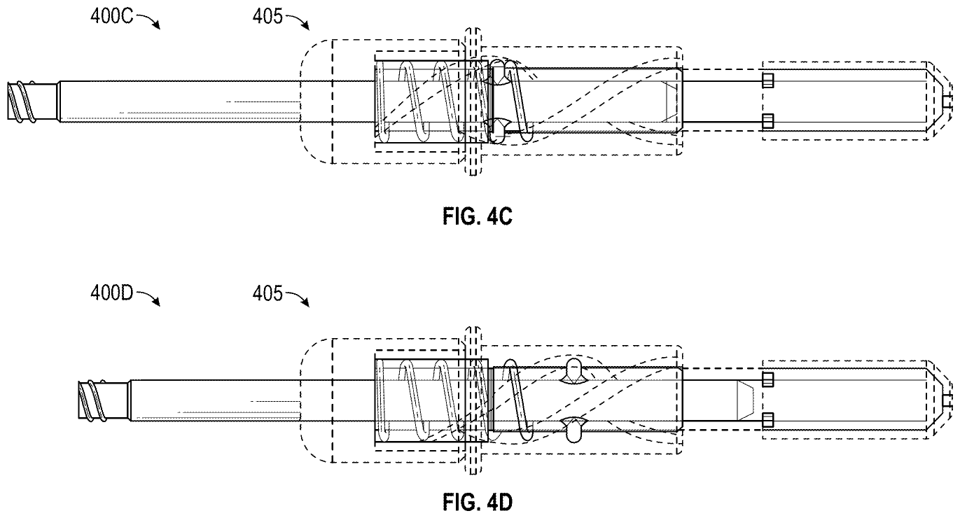

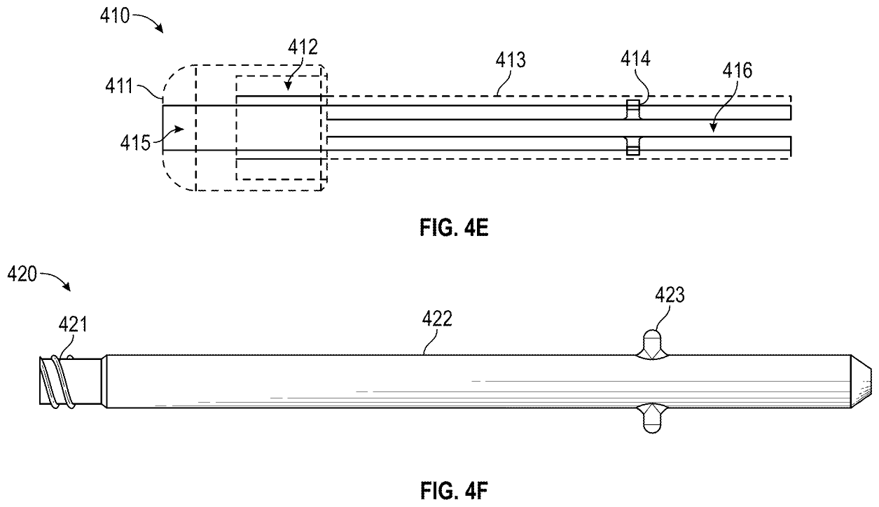

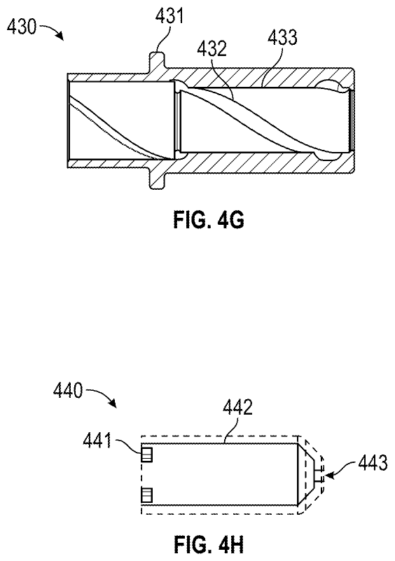

FIGS. 4A-4H illustrate another embodiment of a shaft manipulating handle 4405 as described herein. The handle can be used with any of the tools described herein.

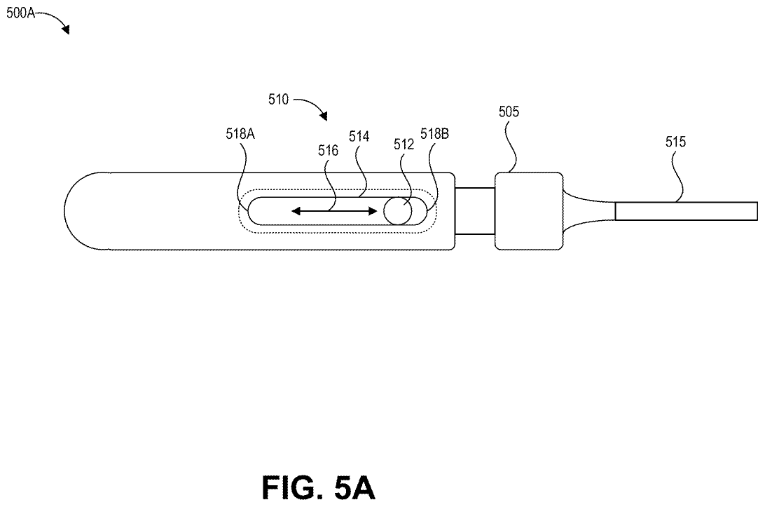

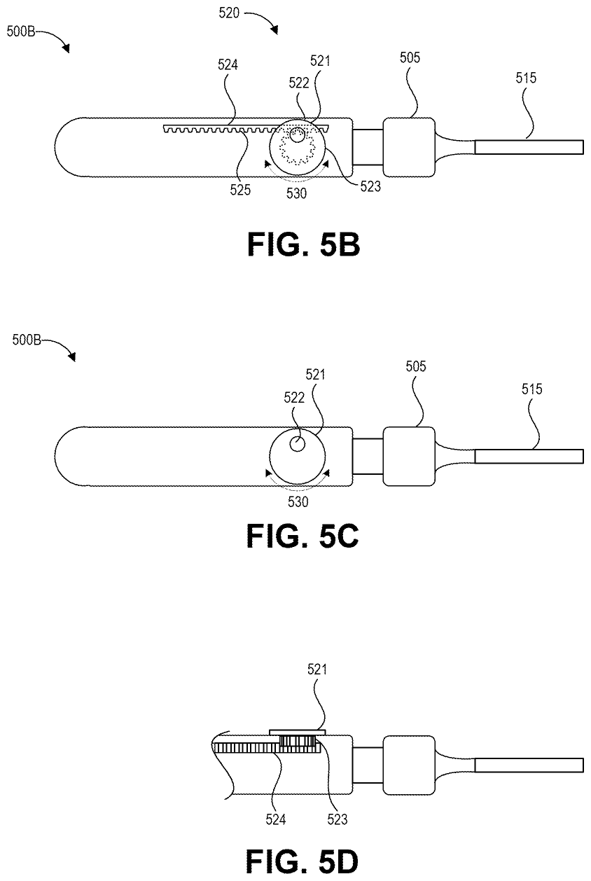

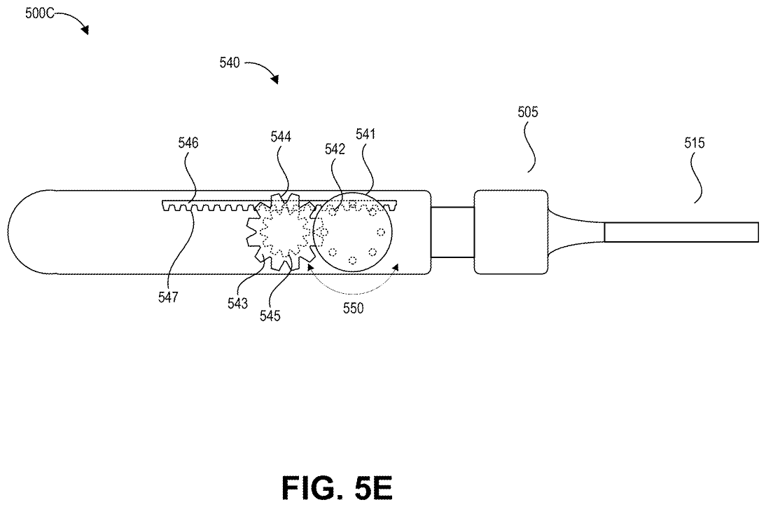

FIGS. 4A-5D illustrate the handle 405 in various states of retraction and extension. FIG. 4A illustrates the handle 405 in a full extension position 400A. In implementations used to drive movement of a biopsy needle through a jacket, for example, the needle would be extended out of the jacket to its maximum extension distance with the handle 405 in the position 400A of FIG. 4A. FIG. 4B illustrates the handle 405 in a retraction position 400B showing the full retraction available via the rotational modality. FIG. 4C illustrates the handle 405 in a full retraction position 400C showing the full retraction available via both the rotational modality and the plunging modality. In the example implementation used to drive movement of the biopsy needle, the needle would be retracted into the jacket to its maximum retraction distance with the handle 405 in the position 400C of FIG. 4C. Upon release of proximally-directed pressure from a grip of the handle, as described more below, the handle 4405 can return to the position 400B of FIG. 4B via force from a biasing element. FIG. 4D illustrates the handle 405 in an intermediate retraction position 400D showing the full retraction available via the plunging modality at an intermediate extension via the rotational modality. FIG. 4D represents one option for dithering a tool into and out of a tissue site in use. Upon release of proximally-directed pressure from the grip of the handle 405 the tool coupled to the handle would be driven distally with force from the biasing element.

FIGS. 4E-4H illustrate the components of the handle 405. FIG. 4E illustrates the base 410 of the handle, FIG. 4F illustrates the shaft 420 of the handle, FIG. 4G illustrates the cam 430 of the handle, and FIG. 4H illustrates the cap 440 of the handle.

Turning to FIG. 4E, the base 410 includes a grip portion 411 having an internal pocket 412, a body 413 having an inner channel 415, side slots 416, and prongs 414. The grip portion 411 can be rotated or plunged by an operator (human or robotic) to actuate the shaft of a medical instrument coupled to the handle. The base 410 provides a supporting structure for the other components of the handle. For example, the body 413 can extend distally from the grip portion 414 and can be formed as two elongate members each having an arc-shaped cross section. These two elongate members can be separated on opposing sides of the base 410 by gaps to form side slots 416. These slots 416 between the elongated members can slidably engage the cross-pin members 423 of the shaft 420 to prevent the shaft 420 from rotating relative to the base 410. The outer surfaces of the elongate members of the body 413 provide an approximately cylindrical surface for slidably engaging the inner surface 433 of the cam 430 during operation of the handle. The internal pocket 412 provides a space for containing a spring or other biasing element that can push against the flange 431 of the cam 430. The inner channel 415 provides an internal cylindrical pathway within which the shaft 420 can move linearly during use. The prongs 414 can provide a locking interface for the cap 440.

Turning to FIG. 4F, the shaft 420 includes an elongated body 422 with a cross-pin feature 423 and an attachment site 421 for fluidically coupling with an aspiration device. Though not illustrated, in some embodiments the shaft 420 can include an interior pathway or lumen, for example to facilitate provision of aspiration through the lumen of an elongate instrument movable via the handle. The shaft 420 of the handle would be operably coupled to the proximal end of the elongate shaft of the medical instrument to drive extension and retraction of the tool coupled to the distal end of the shaft. Thus, linear motion of this shaft is an objective of the handle.

Turning to FIG. 4G, the cam 430 includes flange 431, inner diameter 433, and a helical groove 432 along its internal surface. The groove 432 can be sized to receive the cross-pin of the shaft 420 and to act as a female internal cam interface. This cam interface can have the illustrated helical groove, or can have any spiral profile to achieve the desired linear motion of the shaft for a given amount of twist on the cam 430. Flange 431 can be used as a grip to facilitate the retraction of the plunging motion of the handle and can engage a biasing element in the pocket 412 to drive extension of the plunging motion upon release of proximally-directed pressure from the flange 431.

Turning to FIG. 4H, the cap 440 fastens to prongs 414 of the base 410 via fastening features 441. Thus, the cap 440 provides a physical stop to prevent the cam 430 from moving off the base 410. The cap 440 can include an aperture 443 through which a tubular jacket of an instrument may be passed, as described above.

Not illustrated in FIGS. 4A-4H is a spring or other biasing mechanism that would be placed in the pocket 412 of the base 410 to return the cam 430 to a full-forward position when no proximal linear force is exerted upon it.

FIGS. 5A-5E illustrate various alternate handle embodiments. FIG. 5A illustrates one embodiment of a handle 500A having a jacket 515 extending therefrom and having motion interfaces including a plunging interface 505 and an in-line linear slider 510. As described above, a conduit having a tool at its distal end can be positioned within the jacket 515. In an initial configuration, the distal end of the tool can be positioned at or proximally to the distal end of the jacket. As described above, movement of the tool can be driven by movement of the conduit.

The slider 510 includes a tab 512 slidable within a track 514. The tab 512 can be coupled to an internal drive member that, in turn, is coupled to a proximal end of the conduit. As such, linear motion 516 of the tab 512 can translate 1:1 into extension or retraction of the instrument relative to the distal end of the jacket 515. When the tab 512 is positioned at a proximal end 518A of the track 514 the tool can be in a fully retracted position relative to the jacket 515. When the tab 512 is positioned at the distal end 518A of the track 514 the tool can be in a fully extended position relative to the jacket 515. The tab 512 can be slid to any intermediate position between the proximal and distal ends 518A, 518B and may lock in place. For example, the tab 512 may include a button that, when depressed, allows the sliding motion 516 and that, when released, locks the slider. Although not illustrated, distance markings can be provided along the handle at or near the track 514 to indicate how far the tool is extended.

The plunging interface 505 can be retracted proximally to withdraw an extended tool proximally into the jacket 515 and may be biased to return to the illustrated extended position upon release of force from the plunging interface 505. Thus, the plunging interface 505 can be used to actuate a dithering motion as described above. In some embodiments the plunging interface 505 can extend the tool and be biased toward the retracted position. As the plunging interface 505 is actuated, the tab 512 can slide through the track 514 to provide a visual indication of the extension and/or retraction distance of the tool.

FIGS. 5B-5D illustrate another embodiment of the handle 500B having the jacket 515 and the plunging interface 505, and also having a rack and pinion actuator 520. As described above, the tool and conduit can be positioned at least partly within the jacket 515 and the rack and pinion actuator 520 can drive the fine-control extension and retraction of the tool.

FIG. 5B depicts a cutaway top view of the handle 500B and shows the outline of both the exterior and interior components of the rack and pinion actuator 520, with dashed lines showing the outline of elements positioned behind other elements (from the perspective of the illustrated viewpoint). The rack and pinion actuator 520 includes a rotational wheel 521 having a detent 522 to facilitate grip during rotation by a user. Alternate embodiments could additionally or alternatively include ridges around the outer circumference of the wheel 521.

The wheel 521 is coupled to a gear 523 having a number of radial teeth. These teeth can engage corresponding teeth 525 in a rack 524 to actuate linear motion along the longitudinal axis of the handle 500B in response to rotation 530 of the wheel 521. In some embodiments, the rack 524 can move linearly within the handle 500B and can be coupled to the internal drive member that, in turn, is coupled to the proximal end of the conduit. Thus, movement of the rack 524 can translate 1:1 into extension or retraction of the instrument relative to the distal end of the jacket 515. In other embodiments, the wheel 521 and gear 523 can move linearly along the handle and the gear 523 can be coupled to the internal drive member to actuate the tool extension and retraction.

As shown in the exterior top view depicted in FIG. 5C, the wheel 521 is located on the outside of the handle 500B. As shown in the cutaway side view depicted in FIG. 5D, the gear 523 and rack 524 are positioned within the handle 500B.

Similar to the handle 500A, the plunging interface 505 of the handle 500B can be retracted proximally and/or extended distally to withdraw or extend the tool relative to the jacket 515 and may be biased to return to its initial position upon release of force from the plunging interface 505. Thus, the plunging interface 505 can be used to actuate a dithering motion as described above. As the plunging interface 505 is actuated, the wheel 521 may rotate and/or the entire rack and pinion actuator 520 can move proximally and distally. This can be accompanied by distance markings to provide a visual indication of the extension and/or retraction distance of the tool.

FIG. 5E illustrates another embodiment of the handle 500B having the jacket 515 and the plunging interface 505, and also having an incremental rotation actuator 540. FIG. 5E depicts a cutaway top view of the handle 500C and shows the outline of both the exterior and interior components of the rack and incremental rotation actuator 540, with dashed lines showing the outline of elements positioned behind other elements (from the perspective of the illustrated viewpoint). As described above, the tool and conduit can be positioned at least partly within the jacket 515 and the rack and incremental rotation actuator 540 can drive the fine-control extension and retraction of the tool.

The incremental rotation actuator 540 includes a rotational wheel 541 having a number of spokes 542 extending inwardly (e.g., toward the interior of the handle 500C) from the wheel 541. Though not illustrated, the top or user-facing side of the wheel 541 can have a detent and/or ridges around its circumference to facilitate grip during rotation by a user, similar to the wheel 521 of FIG. 5C.

The incremental rotation actuator 540 also includes a gear 543 and a rack 546. The gear 543 can include a number of first teeth 544 that are engaged by the spokes 542. As the wheel 541 is rotated 550 and a spoke 542 pushes one of the first teeth 544, the gear 543 can also rotate by a predetermined amount corresponding to the number of the first teeth 544 and the number of spokes 542. As the gear 543 rotates, a number of second teeth 545 also rotate. The second teeth 545 can engage the teeth 547 of the rack 546 to move the rack 546 linearly within the handle 500C. The rack 546 can be coupled to the internal drive member that, in turn, is coupled to the proximal end of the conduit. Thus, movement of the rack 524 can translate 1:1 into extension or retraction of the instrument relative to the distal end of the jacket 515.

In some embodiments, ten degrees of rotation can correspond to a 5 mm movement of the tool. Other embodiments can be designed to correlate other degrees of rotation with other movement distances. As such, the handle 500C can provide for movement of the tool in predetermined increments based on the rotation 550 of the wheel 541.

Similar to the handle 500A, the plunging interface 505 of handle 500C can be retracted proximally and/or extended distally to withdraw or extend the tool relative to the jacket 515 and may be biased to return to its initial position upon release of force from the plunging interface 505. Thus, the plunging interface 505 can be used to actuate a dithering motion as described above. As the plunging interface 505 is actuated, the wheel 541 may rotate and/or the entire incremental rotation actuator 540 can move proximally and distally. This can be accompanied by distance markings to provide a visual indication of the extension and/or retraction distance of the tool.

### Overview of Example Robotic Surgical Systems

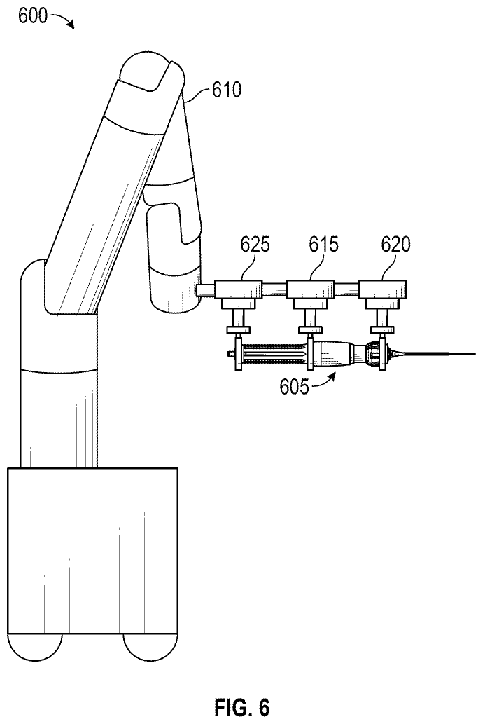

FIG. 6 depicts a schematic diagram of a robotic surgical system 600 for actuating a handle 605 as described herein. Though shown with a particular configuration of the handle 605, any of the described handles can be used with such a system 600. The instrument handle may have a barcode, radio-frequency identifier (RFID), or other suitable identifier to enable the robotic surgical system 600 to identify the handle.

The example robotic system 600 includes an articulated arm 610 configured to locate, and maintain positioning of, the handle 605. At a distal end of the arm 610 are a first grip portion 625 for controlling aspiration or administering therapeutics and two additional grip portions 615, 620 that can open to receive the handle 605 and close around respective portions of the handle 605. The first grip portion 625 can include one or more actuators for gripping and controlling a source of negative (or positive pressure) and/or therapeutics. For example, the first grip portion 625 can include a first actuator for attaching a syringe and a second actuator for robotically controlling a plunger of the syringe.

The second grip portion 615 may maintain a stationary grip and positioning on the handle 605 to provide stability. The third grip portion 620 can be configured to effect the rotational modality of the handle described herein by rotating a wheel or grip of the handle. Further, the third grip portion 620 can be configured to move laterally with respect to the longitudinal axis of the handle to provide the plunging modality described herein. In other embodiments the second grip portion 615 can move to effect the plunging and rotational modalities, alone or in combination with movement of the third grip portion 620. The grip portions 615, 620, 625 can be driven by one or more motors and appropriate actuation mechanisms.

The robotic surgical system 600 is shown with one embodiment of a handle 605 as described herein. Other embodiments of the robotic surgical system 600 can be used to operate variations of the disclosed handle embodiments, for example including different actuation interfaces (e.g., two plunging interfaces, two rotational interfaces, etc.). The robotic surgical system 600 can be configured to control and any or all of the handle actuations, for example the fine control extension/retraction only, dithering only, or both as described above.

The robotic surgical system 600 can include a processor(s) and memory. The memory can store instructions for operation of the various components of the robotic surgical system 600 as well as data produced and used during a surgical procedure. The processor(s) can execute these instructions and process such data to cause operation of the system 600. Although not illustrated, the robotic surgical system 600 can include other components, for example one or more input devices for receiving user input to control motion of surgical instruments (e.g., joysticks, handles, computer mice, trackpads, and gesture detection systems), instrument drivers to effect the motion of the surgical instruments, an additional grip portion for securing and controlling motion of an aspiration device coupled to the handle, a display screen, and the like.

### Overview of Example Methods of Use

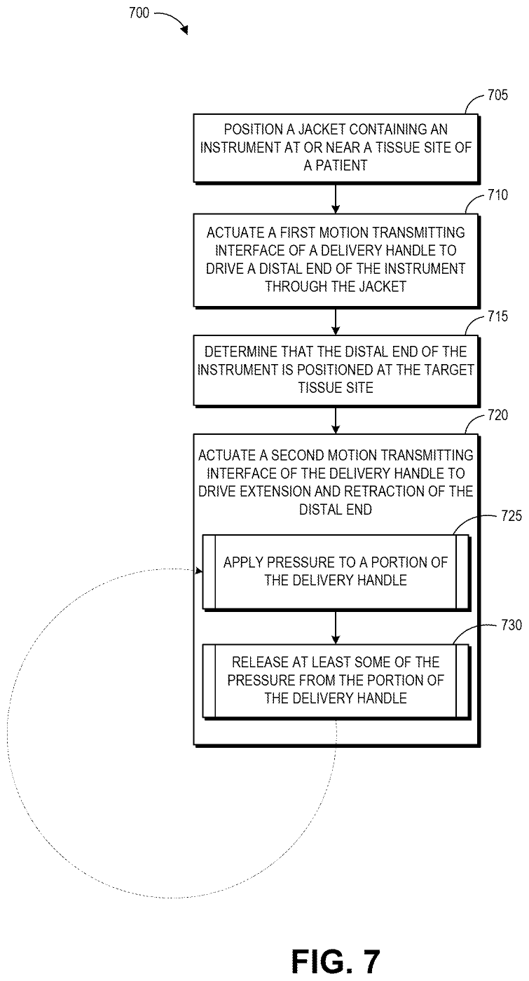

FIG. 7 depicts a flowchart of an embodiment of a process 700 for driving movement of a medical instrument using the handles described herein, for example, handles 105, 205, 305, 405, 500A-500C, and 605, as described above. The process 700 can be implemented by a human operator manually manipulating the handle, a robotic control system operator (such as system 600 described above) mechanically manipulating the handle as directed by a human operator or autonomously, or a combination thereof.

At block 705, the operator (e.g., a human operator or autonomous surgical robot) can position a jacket containing an instrument at or near a tissue site of a patient. As described above, the instrument can be positioned with its distal tip at or near the distal end of the jacket 150 and a conduit 154 or shaft can extend from the proximal end of the tool through the jacket. The jacket can be inserted through the working channel of an endoscope such as a bronchoscope in some embodiments, and the tool can be a needle, brush, forceps, or the like. The conduit can be coupled to a handle 105, 205, 305, 405, 500A-500C, 605 for driving linear motion of the conduit relative to the jacket.

At block 710, the operator can actuate a first motion transmitting interface of the delivery handle 105, 205, 305, 405, 500A-500C, 605 coupled to the instrument to drive the distal end of the instrument to advance through the jacket. As described above and shown in the example of FIG. 1A, this can involve actuation of a rotational modality of the handle, for example by rotational grip 122, 222, 322 or actuation of the motion mechanisms described with respect to handles 500A-500C. Actuation of such a modality can allow the operator to exert fine control over extending the distal tip of the instrument out from the distal end of the jacket. In some procedures, this can involve extending the distal tip of the instrument until it has pierced patient tissue.

At block 715, the operator can determine that the distal end of the instrument is positioned at the target tissue site. In some implementations, a physician may view an image or video of the tissue site via an imaging device at the distal end of an endoscope working channel and may visually confirm that the instrument is positioned at or within the target tissue site. For example, this can be accomplished via fluoroscopy. In some implementations, the physician may view a rendering or model of the positioning of the instrument relative to the patient tissue site to make this determination, for example as output from a robotic bronchoscopy navigation system. In some embodiments block 715 can be performed programmatically via automated image analysis and/or navigation.

At block 720, the operator can actuate a second motion transmitting interface of the delivery handle 105, 205, 305, 405, 500A-500C, 605 to drive extension and retraction of the distal end of the instrument. As described above and shown in the example of FIG. 1B, this can involve actuation of a plunging modality, for example by plunging grip 122, 222, 322.

As shown by sub-blocks 725 and 730, actuation of the second motion transmitting interface can involve a first step of retraction and a second step of extension. At block 725, the operator can apply pressure to a portion of the delivery handle to (1) compress a biasing element of the second motion transmitting interface, and (2) drive retracting motion of the instrument to withdraw the distal end of the instrument from the tissue site. At block 730, the operator can release at least some of the pressure from the portion of the delivery handle to allow expansion of the second motion transmitting interface to drive the distal end of the into the tissue site. In other embodiments, the handle can be structured such that application of pressure results in extension of the tool and release of the pressure retracts the tool. Repetition of blocks 725 and 760 can generate a dithering motion of the tool through repeated extension and retraction which, as described above, may be beneficial in tissue sampling.

After completion of the process 700 the instrument can be withdrawn back into the jacket, for example via the first motion transmitting interface, and the jacket can be withdrawn from the patient tissue site. Any obtained sample can be expelled from the instrument for the desired analysis.

### Example Handles for Expelling Samples

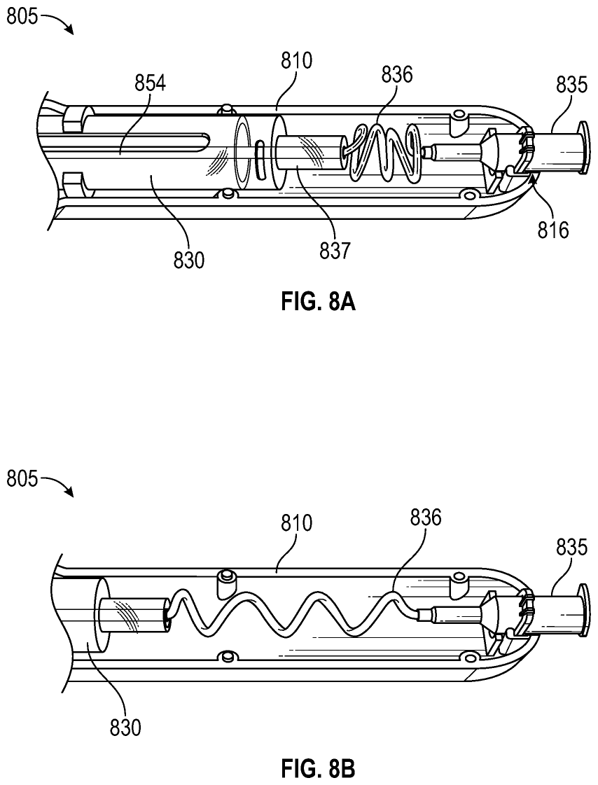

FIGS. 8A and 8B illustrate cutaway views of a proximal end of a handle 805, such as the handle 105 of the instrument 100 illustrated in FIGS. 1A-1F. In particular, FIG. 8A illustrates the handle 805 when a tool, such as, e.g., a biopsy needle or tool 156 illustrated in FIGS. 1A-1F, is in a fully retracted position and FIG. 8B illustrates the handle 805 when the tool is in a fully extended position.

With reference to FIGS. 8A and 8B, the handle 805 includes a casing 810 having a proximal aperture 816 at the proximal end of the handle 805. A fluid fitting 835 is coupled to the casing 810, for example, by placing the fluid fitting 835 in the proximal aperture 816. The casing 810 houses a handle member 830 forming a recess 837 in which a conduit 854 is secured. The conduit 854 may be attached to the tool (not illustrated) configured to be extended from and retracted into the distal end of an elongate channel (e.g., a lumen of the jacket 150 of FIGS. 1A-1F) based on movement of the handle member 830 with respect to the casing 810. The handle member 830 can be positioned at least partly within an internal volume of the casing 810. The handle member 830 is coupled to the tool and configured to move along with respect to the casing 810 (e.g., along the longitudinal axis of the casing 810) as the tool is moved between the retracted position and the extended position.

A flexible tube 836 passes through the conduit 854 and is fluidly connected to the fluid fitting 835. In detail, the flexible tube 836 can form a portion of a lumen extending between the fluid fitting 835 and a distal end of the tool. For example, the flexible tube 836 can extend from the fluid fitting 835 to the distal end of the tool by passing though the conduit 854. In some embodiments, the flexible tube 836 can comprise a length of coiled tubing. The flexible tube 836 provides a flexible fluid path that accommodates linear motion of the handle member 830 with respect to the casing 810. For example, the flexible tube 836 can be implemented as coiled HDPE tubing, and, in some embodiments, this can form a portion of the conduit 854 positioned proximally relative to the bonding recess 837. As shown in FIGS. 8A and 8B, the flexible tube 836 can include a plurality of coils that are more closely spaced when the tool is in a retracted position (e.g., as shown in FIG. 8A) and are more spaced apart when the tool is in an extended position (e.g., as shown in FIG. 8B). The plurality of coils can be formed by curing or shape-setting a length of the tubing into a coiled pattern that permits extension and retraction of the flexible tubing without kinking or tangling the tubing. Such a shape of multiple coils can be formed using a length of tubing that is much longer than the distance between the handle member 930 and the fluid fitting 935 when the handle member 930 is in the fully extended position. A sleeve of polyolefin heat shrink can be used to secure the coiled tubing of the flexible tube 836 to the fluid fitting 835 in some implementations. The polyolefin heat shrink can assist in forming a thermal bond between the flexible tube 836 and the fluid fitting 835.

A syringe or other aspiration or respiration device can be attached to the fluid fitting 835 to provide a vacuum to aid in drawing tissue from a target into the tool and/or provide pressure to the sample when expelling the sample for analysis (e.g., expelling the sample onto a slide). The medical device can be used multiple times during a medical procedure, for example, to take samples from a plurality of targets. In certain circumstances, it may not be possible to expel the samples from the tool, for example, due to the tissue samples being tightly packed in the needle. If the samples cannot be retrieved from the tool, it may be necessary to cut or otherwise destroy the tool.

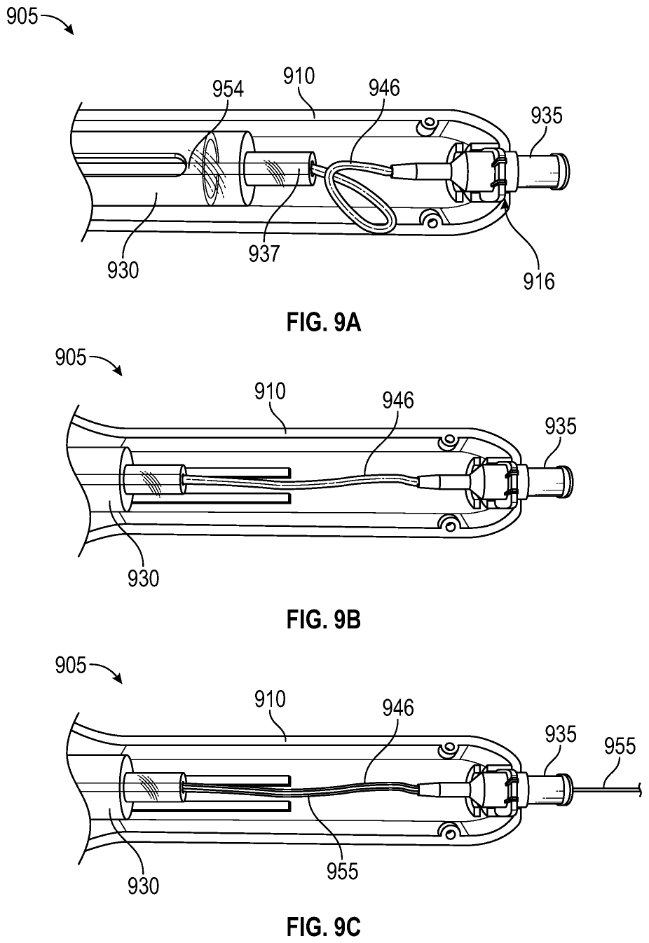

FIGS. 9A-9C illustrate cutaway views of another embodiment of a proximal end of a handle 905, such as a modified embodiment of the handle 105 of the instrument 100 illustrated in FIGS. 1A-1F in accordance with aspects of this disclosure. In particular, FIG. 9A illustrates the handle 905 when a tool, such as, e.g., a biopsy needle or tool 156 illustrated in FIGS. 1A-1F, is in a fully retracted position, FIG. 9B illustrates the handle 905 when the tool is in a fully extended position, and FIG. 9C illustrates a stylet 955 being inserted into the handle 905 which can be used to expel samples which are stuck in the tool.

Similar to the embodiment illustrated in FIGS. 8A and 8B, the proximal end of the handle 905 in the embodiment of FIGS. 9A-9C includes a casing 910 having a proximal aperture 916, a fluid fitting 935 coupled to the casing 910, a handle member 930 forming a recess 937 in which a conduit 954 is secured, and a flexible tube 946 fluidly connecting the tool to the fluid fitting 935. The fluid fitting 935 and the flexible tube 946 passing through the conduit 954 together form a central lumen with a single continuous fluid path between the fluid fitting 935 and the tool. This fluid path allows changes in air pressure (e.g., via the use of a syringe attached to the fluid fitting) to collect and/or expel tissue samples.

Similar to the embodiment of FIGS. 8A and 8B, the flexible tube 946 allows for extension and retraction of the tool into an elongate channel (e.g., a lumen of the jacket 150 of FIG. 1A-1F) while maintaining the closed fluid path of the lumen.

In the embodiment of FIG. 9A-9C, the flexible tube 946 is configured to allow a stylet 955 to pass through the lumen to the distal end of the tool when the tool is in the extended position. FIG. 9C illustrates the stylet 955 inserted into the lumen formed by the flexible tube 946 such that the stylet 955 can be passed through the lumen formed by the flexible tube 946 and the conduit 954 to the distal end of the tool to physically expel samples from the distal end of the tool. The medical device can therefore use the stylet 955 as an auxiliary method to physically expel the samples from the tool, e.g., when pressure from the syringe is insufficient to expel the samples.

In contrast to the handle 805 of FIGS. 8A and 8B, the flexible tube 946 in the embodiment of FIGS. 9A-9C is configured to be substantially straight when the tool is in the extended position. By removing the tight turns associated with the coiled flexible tube 836 as shown in FIG. 8B, the substantially straight flexible tube 946 of FIGS. 9B and 9C allows the stylet 955 to be inserted through the fluid fitting 935, the flexible tube 946, and the conduit 954 without catching on any coils formed by the coiled flexible tube 946, which could potentially tear or pierce the flexible tube 946. The flexible tube 946 is also configured to form a single loop when the tool is in the fully retracted position as shown in FIG. 9A. The single loop can result from a curling of the flexible tube 946 when retracting from a substantially straight configuration when in the fully extended position to the fully retracted position. In some implementations, such a single loop can naturally form without a need for shape-setting or biasing the flexible tube into a coiled pattern, due to the length of the flexible tube being the same as or only slightly longer than the distance between the handle member and the fluid fitting in the fully extended position. Although only one loop is illustrated, the flexible tube 946 may be configured to form two or more loops in other embodiments.

In certain implementations, the flexible tube 946 is flexible enough to allow the stylet 955 to traverse the flexible tube 946 and push any stuck samples out of the tool. In addition, the flexible tube 946 can be configured to have a defined degree of slack, while still remaining substantially straight, when the tool is in the extended position so as to reduce stress applied by the flexible tube 946 to the fluid fitting 935 and the handle member 930. In some implementations, the length of the flexible tube 946 is slightly longer than the distance between the handle member 930 and the fluid fitting 935 when the tool is in the extended position by, e.g., about 0.5 mm to about 5 mm, or by, e.g., about 1 mm to about 2 mm. For example, if the flexible tube 946 had a length that is less than the distance between the handle member 930 and the fluid fitting 935 when the tool is in the extended position, the flexible tube 946 may exert forces on handle member 930 and the fluid fitting 935 due to stretching of the flexible tube 946. Making the length of the flexible tube 946 only slightly longer than the distance between the handle member 930 and fluid fitting 935 in the fully extended position allows the flexible tube 946 to maintain a substantially straight configuration that facilitates insertion of stylet 955 when in the fully extended position.

In some implementations, the stylet 955 can be formed of steel. However, the stylet 955 is not limited to being formed of steel and any material may be used for the stylet as long as the stylet 955 has sufficient longitudinal strength to be passed through the lumen in the medical instrument and used to expel stuck samples from the tool. The stylet 955 can include a bullnose or full rounded tip to help the stylet 955 traverse the lumen including transitions in the lumen, for example, within the fluid fitting 935.

In some implementation, some physicians may choose to navigate the medical instrument through the patient's luminal network while a stylet 955 is placed in the lumen. By placing the stylet 955 in the lumen up to the distal end of the tool, the stylet 955 can help prevent unwanted tissue or bodily fluids from entering the tool while driving the tool through the luminal network, for example, by physically blocking the opening in the end of the tool. After the tool has been advance to the target, the physician can remove the stylet 955 from the medical instrument, attach an aspiration or respiration device (e.g., a syringe) to the fluid fitting 935, and take samples of the target using the tool. Since the tool is typically in a retracted position while the medical instrument is being driven through the luminal network (e.g., to prevent the sharp distal end of the tool from injuring the patient), the stylet 955 should be flexible enough to allow the handle member 930 to be retracted towards the proximal end of the casing 910 as shown in FIG. 9A. In some implementations, the stylet 955 can be formed of nitinol, which may provide the stylet 955 with sufficient flexibility to allow the handle member 930 to be retracted towards the proximal end of the casing 910 to retract the tool into the distal end of the elongate member.

In some implementations, the nitinol stylet 955 may be flexible such that the nitinol stylet can be passed through the lumen to the distal end of the tool when the tool is in a position between the extended position and the retraced position (e.g., an intermediate position between FIGS. 9A and 9B). Once the nitinol stylet 955 has been inserted into the lumen, the tool can be retracted into the fully retracted position and the nitinol stylet 955 can be bent along with the flexible tube 946, for example, into a loop. A physician can then drive the tool into a patient's luminal network with the nitinol style 955 inserted into the lumen in order to prevent unwanted tissue or bodily fluids from entering the distal end of tool as described above.

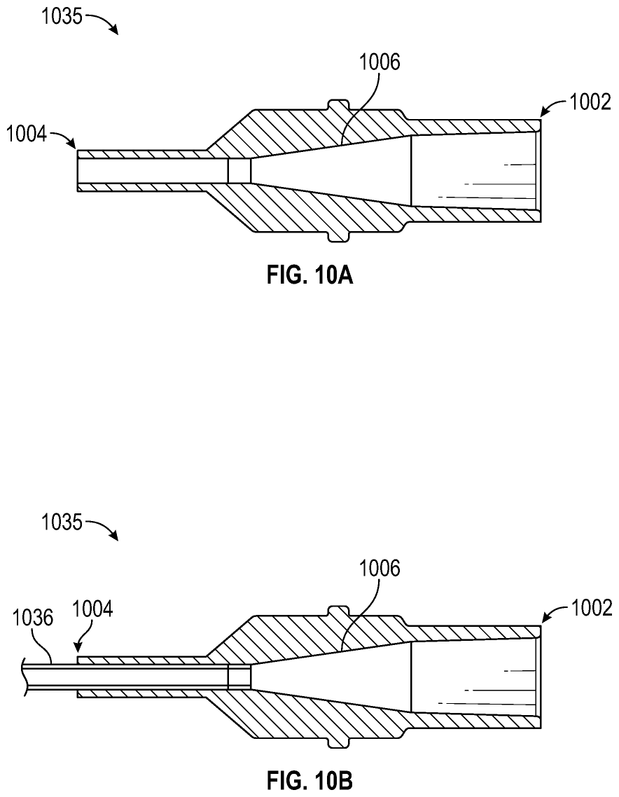

FIGS. 10A and 10B illustrate cutaway views of an embodiment of a fluid fitting 1035, such as the fluid fitting 835 of FIGS. 8A and 8B. In particular, FIG. 10A illustrates a cutaway view of the fluid fitting 1035 itself and FIG. 10B illustrates a cutaway view of the fluid fitting 1035 connected to a flexible tube 1036. With reference to FIGS. 10A and 10B, the fluid fitting 1035 has a proximal end 1002 and a distal end 1004. An aspiration or respiration device (e.g., a syringe) can be attached to the proximal end 1002 of the fluid fitting 1035. The fluid fitting 1035 also has an inner surface 1006 which, in one example, can taper from the proximal end 1002 to the distal end 1004.

When coupled to the flexible tube 1036, the flexible tube 1036 can be inserted into the distal end 1004 of the fluid fitting 1035. The flexible tube 1036 may be hermetically sealed to the fluid fitting 1035 such that a vacuum or air pressure generated by the syringe can be communicated to the distal end of tool via the lumen partially formed with the flexible tube 1036.

In the embodiment of FIG. 10B, the flexible tube 1036 may extend into or past the tapered section of the inner surface 1006 of the fluid fitting 1035. Since the inner diameter of the flexible tube 1036 is less than the diameter of the tapered section of the inner surface 1006 of the fluid fitting 1035, in some instances it may be difficult to insert a stylet into the flexible tube 1036 via the fluid fitting 1035.

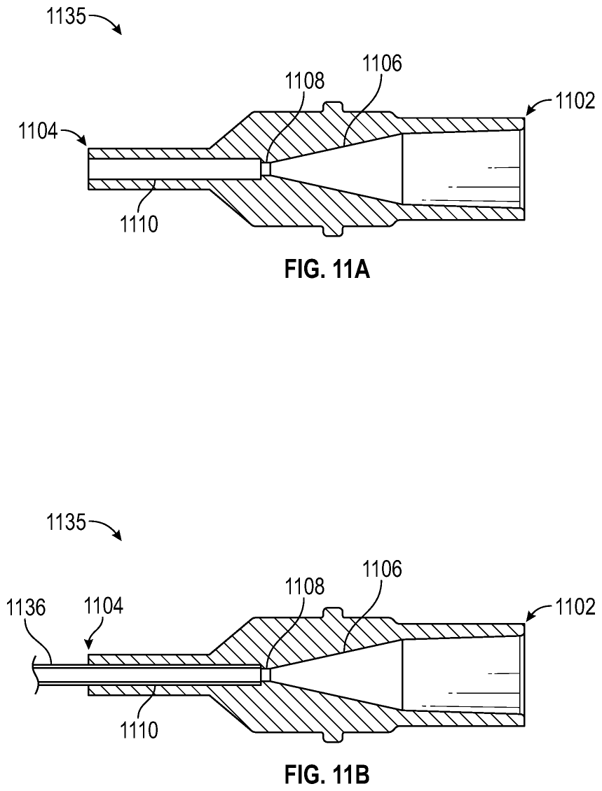

FIGS. 11A and 11B illustrate cutaway views of another embodiment of a fluid fitting 1135, such as the fluid fitting 935 of FIGS. 9A-9C which can guide a stylet into a flexible tube 1136. In particular, FIG. 11A illustrates a cutaway view of the fluid fitting 1135 itself and FIG. 11B illustrates a cutaway view of the fluid fitting 1135 connected to the flexible tube 1136. With reference to FIGS. 11A and 11B, the fluid fitting 1135 has a proximal end 1102 and a distal end 1104 and an aspiration or respiration device (e.g., a syringe) can be attached to the proximal end of the fluid fitting 1135.

The fluid fitting 1135 has an inner surface 1106 which can taper from the proximal end 1002 to the distal end 1004. As shown in FIG. 11B, the fluid fitting 1135 can be coupled to a flexible tube 1136 at the distal end 1104 of the fluid fitting 1135. The inner surface 1106 can be shaped to guide a stylet into the flexible tube 1136, for example, the inner surface 1106 of the fluid fitting 1135 can have a conical shape to aid in guiding the stylet into the flexible tube 1136.

In some embodiments, the inner surface 1106 of the fluid fitting 1135 tapers to an inner diameter that is less than an inner diameter of the flexible tube 1136. For example, the inner surface 1106 can tapers from a first inner diameter at the proximal end 1102 of the fluid fitting 1135 to a second inner diameter at a constricted point 1108 distal from the proximal end 1102 of the fluid fitting 1135. The inner surface 1106 of the fluid fitting 1135 can also define a flexible tube accommodation section 1110 into which the proximal end of the flexible tube 1136 can be inserted. The flexible tube 1136 can abut the proximal end of the flexible tube accommodation section 1110 so as to be substantially flush with the constricted point 1108 of the inner surface 1106.

In certain embodiments, the second inner diameter of the fluid fitting 1135 is not greater than the inner diameter of the flexible tube 1136 such that the stylet can be inserted into the flexible tube from the constricted point 1108 without catching on a proximal end surface of the flexible tube. For example, the first inner diameter of the fluid fitting 1106 can be greater than the inner diameter of the flexible tube 1136.

Embodiments of this disclosure provide a number of distinct advantages over other configurations. Specifically, by allowing a stylet to be passed through the flexible tube, a use can remove samples from a distal end of the tool using alternate method, without having to destroy the tool. Embodiments of this disclosure can be implemented without any change to the functionality of the device during sample collection. For example, the medical instrument can be configured to allow a stylet to pass through the central lumen by incorporating the flexible tube and/or fluid fitting design of FIGS. 9A-11B. The device can continue to be used to take further samples (e.g., from addition targets within a luminal network) after the stylet has been used to expel or dislodge the stuck or embedded samples.

### Implementing Systems and Terminology

Implementations disclosed herein provide systems, methods and apparatus for actuating extension and retraction of a remotely-disposed instrument by way of linear motion of a shaft of the instrument secured within a handle.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The robotic motion actuation functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

What is claimed is:

1. A medical instrument, comprising:
an elongate channel having a distal end;
a tool configured to be extended from and retracted into the distal end of the elongate channel; and
a handle configured to drive movement of the tool between a retracted position and an extended position, the handle comprising:
a casing,
a handle member coupled to the tool and configured to move with respect to the casing as the tool is moved between the retracted position and the extended position,
a fluid fitting coupled to the casing, and
a flexible tube connecting the fluid fitting to the handle member and forming a portion of a lumen extending between the fluid fitting and a distal end of the tool, the flexible tube configured to allow a stylet to pass through the lumen to the distal end of the tool when the tool is in the extended position.

2. The medical instrument of claim 1, wherein the flexible tube is configured to be substantially straight when the tool is in the extended position and the flexible tube is configured to form at least one loop when the tool is in the retracted position.

3. The medical instrument of claim 2, wherein the flexible tube is configured to have a defined degree of slack when the tool is in the extended position so as to reduce stress applied by the flexible tube to the fluid fitting and the handle member.

4. The medical instrument of claim 1, wherein the flexible tube is configured to form a single loop when the tool is in the retracted position.

5. The medical instrument of claim 1, wherein a length of the flexible tube is 0.5-5 mm longer than a distance between the handle member and the fluid fitting when the tool is in the extended position.

6. The medical instrument of claim 1, further comprising:
an actuation sleeve positioned at least partly within a distal portion of the casing and configured to drive the movement of the tool and the handle member, and
a biasing element coupled between the casing and the handle member.

7. The medical instrument of claim 6, wherein:
the actuation sleeve comprises a rotation grip and a plunger grip,
the handle is configured to translate rotation of the rotation grip into linear motion of the handle member, and
the biasing element is positioned to compress as the plunger grip is retracted such that, upon release of the plunger grip, a bias of the biasing element drives linear motion of the handle member.

8. The medical instrument of claim 1, wherein the fluid fitting has a conical inner surface configured to guide the stylet into the flexible tube.

9. The medical instrument of claim 1, wherein:
the flexible tube has an inner diameter,
the flexible tube is connected to a distal end of the fluid fitting,
the fluid fitting has an inner surface that tapers from a first inner diameter to a second inner diameter at a point distal from the first inner diameter of the fluid fitting,
the second inner diameter of the fluid fitting is not greater than the inner diameter of the flexible tube, and
wherein the first inner diameter of the fluid fitting is greater than the inner diameter of the flexible tube.

10. The medical instrument of claim 1, wherein the stylet comprises a steel stylet.

11. The medical instrument of claim 1, wherein the stylet comprises a nitinol stylet, and the flexible tube is further configured to allow the nitinol stylet to pass through the lumen to the distal end of the tool when the tool is in a position between the extended position and the retracted position.

12. The medical instrument of claim 11, wherein the handle is further configured to drive movement of the tool to the retracted position while the nitinol stylet is positioned within the lumen.

13. The medical instrument of claim 1, wherein the tool comprises a needle configured to take a biopsy sample.

14. The medical instrument of claim 13, wherein the fluid fitting is configured to couple to an aspiration device or a respiration device configured to expel the biopsy sample from the needle.

15. A handle of a medical instrument, the handle comprising:
a casing having a proximal end;
a handle member positioned at least partly within the casing, the handle member coupled to a needle and configured to move with respect to the casing as the needle is moved between a retracted position and an extended position;
a fluid fitting at the proximal end of the casing; and
a flexible tube connecting the fluid fitting to the handle member and forming a portion of a lumen extending between the fluid fitting and the needle, the flexible tube configured to allow a stylet to pass through the lumen to the distal end of the needle when the needle is in the extended position.

16. The handle of claim 15, wherein the flexible tube is configured to be substantially straight when the needle is in the extended position and the flexible tube is configured to form at least one loop when the needle is in the retracted position.

17. The handle of claim 16, wherein the flexible tube is configured to have a defined degree of slack when the needle is in the extended position so as to reduce stress applied by the flexible tube to the fluid fitting and the handle member.

18. The handle of claim 15, further comprising:
an actuation sleeve positioned at least partly within a distal portion of the casing and configured to drive the movement of the needle and the handle member, and
a biasing element coupled between the casing and the handle member.

19. The handle of claim 18, wherein:
the actuation sleeve comprises a rotation grip and a plunger grip,
the handle is configured to translate rotation of the rotation grip into linear motion of the handle member, and
the biasing element is positioned to compress as the plunger grip is retracted such that, upon release of the plunger grip, a bias of the biasing element drives linear motion of the handle member.

20. The handle of claim 15, wherein the fluid fitting has a conical inner surface configured to guide the stylet into the flexible tube.

21. The handle of claim 15, wherein:
the flexible tube has an inner diameter,
the flexible tube is connected to a distal end of the fluid fitting,
the fluid fitting has an inner surface that tapers from a first inner diameter to a second inner diameter at a point distal from the first inner diameter of the fluid fitting, and
the second inner diameter of the fluid fitting is not greater than the inner diameter of the flexible tube.

22. The handle of claim 21, wherein the first inner diameter of the fluid fitting is greater than the inner diameter of the flexible tube.

23. The handle of claim 15, wherein the needle is configured to take a biopsy sample.

24. The handle of claim 23, wherein the fluid fitting is configured to couple to an aspiration device or a respiration device configured to expel the biopsy sample from the needle.

25. A medical instrument, comprising:
an elongate channel having a distal end;
a tool configured to be extended from and retracted into the distal end of the elongate channel; and
a handle configured to drive movement of the tool between a retracted position and an extended position, the handle comprising:
a casing,
a handle member positioned at least partly within the casing, the handle member coupled to the tool and configured to move in a longitudinal direction with respect to the casing as the tool is moved between the retracted position and the extended position, and
a flexible tube connecting the casing to the handle member and forming a portion of a lumen extending between the casing and the tool, the flexible tube configured to be substantially straight when the tool is in the extended position and the flexible tube is configured to form at least one loop when the tool is in the retracted position.

26. The medical instrument of claim 25, wherein the flexible tube configured to allow a stylet to pass through the lumen to the distal end of the tool when the tool is in the extended position.

27. The medical instrument of claim 25, wherein the flexible tube is configured to have a defined degree of slack when the tool is in the extended position so as to reduce stress applied by the flexible tube to the fluid fitting and the handle member.

28. The medical instrument of claim 25, wherein the flexible tube is configured to form a single loop when the tool is in the retracted position.

29. The medical instrument of claim 25, wherein a length of the flexible tube is 0.5-5 mm longer than a distance between the handle member and the fluid fitting when the tool is in the extended position.

30. The medical instrument of claim 25, wherein the handle further comprises:
an actuation sleeve positioned at least partly within a distal portion of the casing and configured to drive the movement of the tool and the handle member, and
a biasing element coupled between the casing and the handle member.
